(19) United States
(12) Patent Application Publication
(10) Pub. No.: US 2008/0033518 A1
(43) Pub. Date: Feb. 7, 2008

(54) Thermoregulating units

(75) Inventors: Benny Rousso, Rishon-LeZion (IL); Yoram Izhaki, Moshav Kfar Hess (IL); Or Shabtay, Doar-Na Bikat Beit HaKerem (IL)

Correspondence Address: Martin D. Moynihan;PRTSI, Inc., P.O. Box 16446, Arlington, VA, 22215, US

(73) Assignee: Tylerton International Inc., Tortola (VG)

(21) Appl. No.: 11/882,549

(22) Filed: Aug. 2, 2007

Related U.S. Application Data
(63) Continuation-in-part of application No. PCT/IL07/00136, filed on Feb. 1, 2007.
(60) Provisional application No. 60/764,398, filed on Feb. 2, 2006.

(51) Int. Cl. A61B 18/02

(52) U.S. Cl. 607/112

(57) ABSTRACT

A thermoregulating unit for increasing metabolism of at least a portion of a human body, the thermoregulating unit comprises a support surface configured for supporting at least a portion of a human body, and at least one thermoregulating chamber juxtaposed on said support surface, said at least one thermoregulating chamber configured to draw heat away from said at least a portion of the human body supported on said support surface.

### RELATED APPLICATIONS

This application is a continuation-in-part of PCT Patent Application No. PCT/IL2007/000136 filed Feb. 1, 2007, which claims the benefit of U.S. Provisional Patent Application No. 60/764,398 filed Feb. 2, 2006, the disclosures of both are incorporated herein by reference.

### FIELD AND BACKGROUND OF THE INVENTION

The present invention relates to thermoregulation units for increasing the metabolism rate of in a human body.

Obesity is becoming a widespread problem in developed countries. Some common solutions for obesity include exercise and diet regimens. While on the short-term exercise and diet can be effective in reducing weight, on the long term the regimens are often neglected, resulting in the subject failing to attain a healthy weight or even regaining previously lost weight.

Increasing the metabolic output of the body by reducing body temperature has been shown to have a potential for causing weight loss. For example, during exposure to the cold, shivering can cause up to a 2.5 times increase in metabolic rate in an effort to maintain core temperature; which may facilitate weight loss.

“Fat Loss in the Cold?”; Ellen Glickman-Weiss at VirtualMuscle.com, referencing “Thermoregulatory Thermogenesis of Humans During Cold Stress”; I. Jacobs, L. Martineau et al; “Exercise and Sports Science Reviews”, 1993; the disclosure of which is hereby incorporated herein by reference.

### SUMMARY OF THE INVENTION

According to one aspect of the invention, there is provided, a thermoregulating unit for increasing metabolism of at least a portion of a human body, the thermoregulating unit comprising a support surface configured for supporting at least a portion of a human body, and at least one thermoregulating chamber juxtaposed on the support surface, the at least one thermoregulating chamber containing a fluid configured to draw heat away from the at least a portion of the human body supported on the support surface.

In embodiments, the at least one thermoregulating chamber passively draws heat away from the at least a portion of the human body supported on the support surface.

In embodiments, the at least one thermoregulating chamber is removably attached to the support surface.

In embodiments, the at least one thermoregulating chamber is configured to be cooled in a cooling unit.

In embodiments, the at least one thermoregulating chamber is configured to be frozen in a freezer.

In embodiments, the thermoregulating unit includes at least one of: one seat, one backrest, one headrest, one armrest, one hand rest, one leg rest, and one footrest.

In embodiments, the at least one chamber is contained in at least one of the: one seat, one backrest, one headrest, one armrest, one hand rest, one leg rest, and one footrest.

In embodiments, wherein the support surface comprises at least two of the: one seat, one backrest, one headrest, one armrest, one hand rest, one leg rest, and one footrest.

In embodiments, the support surface is configured to draw heat away from the at least a portion of the supported human body without exceeding a chosen tolerable level of discomfort.

In embodiments, the thermoregulating unit includes a pump having an inlet and an outlet configured to circulate cooling fluid through an active cooling chamber containing the cooling fluid and connected to the inlet and outlet.

In embodiments, the active cooling chamber passes through at least a portion of at least one of the: one seat, one backrest, one headrest, one armrest, one hand rest, one leg rest, and one footrest.

In embodiments, the thermoregulating unit includes a refrigeration unit connected to the active cooling chamber, the refrigeration unit configured to cool the cooling fluid when the cooling fluid passes therethrough.

In embodiments, the thermoregulating unit includes an acoustic insulating container at least partially surrounding at least one of the: refrigeration unit and pump.

In embodiments, the thermoregulating unit includes a noise canceling wave generator operatively associated with at least one of the: refrigeration unit and pump.

In embodiments, the thermoregulating unit includes a controller adapted to actively control a rate at which the pump circulates the cooling fluid.

In embodiments, the thermoregulating unit includes at least one sensor connected to the controller, the sensor measuring the temperature in the at least one body portion.

In embodiments, the controller additionally regulates at least one of: a rate of heat loss, and a level of user discomfort.

In embodiments, the refrigeration unit comprises at least one of: a Peltier unit, and an evaporative cooling unit.

In embodiments, the thermoregulating unit includes a heating element configured to raise the temperature of the cooling fluid to regulate the rate of heat loss, or the level of discomfort, or both.

In embodiments, the thermoregulating unit includes one vibrator operatively associated with the unit, the one vibrator configured to cause frictional interaction between the portion of the human body and the support surface, thereby causing vasodilation that increases a rate of heat loss.

In embodiments, the unit is configured for use in a vehicle. In embodiments, the at least one thermoregulating chamber includes a foam material.

In embodiments, the foam material has a thickness of at least between about 10 and 30 centimeters.

In alternative embodiments, the material has a thickness of no more than between about 31 and 100 centimeters.

In embodiments, the at least one thermoregulating chamber includes thermoregulating materials from the group of materials consisting of: metal pads, liquid capsules, diamond chips, heat pipes, mega flats, and silicon pads.

In embodiments, the at least one thermoregulating chamber includes a fan configured to foster heat exchange between the thermoregulating material and the environment.

In embodiments, the at least one thermoregulating chamber includes a fluid from the group of fluids consisting of: a phase change fluid, carbon dioxide, nitrous oxide, a chemically reactive fluid that cools during a chemical reaction, and a chemically reactive fluid that heats during a chemical reaction.

In embodiments, the at least one thermoregulating chamber includes a chamber circulation pump that circulates the fluid contained therein.

In embodiments, the support surface comprises a material that facilitates removal of heat from a body selected from the group of materials consisting of: polyethylene, polyvinyl chloride, polyurethane, nylon and a biocompatible polymer fiber.

In embodiments, the material has a thickness of at least about between about 1.0 and 6.0 millimeters.

In embodiments, the material has a thickness of no more than between about 7.0 and 15.0 millimeters.

In embodiments, the material comprises a material that facilitates removal of heat from a body selected from the group of materials consisting of: nitinol, stainless steel shape memory materials, metals, synthetic biostable polymer, a natural polymer, and an inorganic material.

In embodiments, the biostable polymer comprises a material from the group of materials consisting of: a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, a polyester, an aromatic polyester, a polysulfone, and a silicone rubber.

In embodiments, the natural polymer comprises a material from the group of materials consisting of: a polyolefin, a polyurethane, a Mylar, a silicone, and a fluorinated polyolefin.

In embodiments, the material comprises a material having a property from the group of properties consisting of: compliant, flexible, plastic, and rigid.

In embodiments, the material includes an API (active pharmaceutical ingredient) that biologically aids in increasing metabolism of a user when the user is nude or wearing garments configured to absorb the API.

In embodiments, the API comprises a chemotherapeutic selected from the group consisting of: peptides, proteins, calcitonin, analgesics, antidepressants, antihistamines, anti-inflammatory agents, antiirritants, antilipemics, antipruritics, non-steroidal anti-inflammatory agents, vasodilators, and mixtures thereof.

In embodiments, the API comprises an analgesic selected from the group consisting of: benzocaine, butamben picrate, dibucaine, dimethisoquin, dyclonine, lidocaine, pramoxine, tetracaine, salicylates and derivatives, esters, salts, and mixtures thereof.

In embodiments, the material is formed by a process from the group of processes consisting of: knitting, braiding, knotting, wrapping, interlacing, and electrospinning.

According to another aspect of the invention, there is provided, a thermoregulating unit for increasing metabolism of at least a portion of a human body, the thermoregulating unit comprising a support surface configured for supporting at least a portion of a human body, at least one cooling insert configured to be cooled in an ancillary cooling device, the at least one cooling insert configured to be inserted into the support surface and draw heat away from the at least a portion of the human body supported on the support surface.

In embodiments, the at least one cooling insert contains at least one of: a phase change fluid, carbon dioxide, nitrous oxide, a chemically reactive fluid that cools during a chemical reaction, and a chemically reactive fluid that heats during a chemical reaction.

In embodiments, the thermoregulating unit includes at least one inflatable cushion.

In embodiments, the at least one inflatable cushion is juxtaposed over the at least one cooling insert.

In embodiments, the thermoregulating unit includes at least one thermoregulating chamber thermally coupled to the at least one cooling insert and configured to transfer heat to the at least one cooling insert.

In embodiments, the at least one thermoregulating chamber is thermally coupled to the supported body and additionally configured to draw heat away from the at least a portion of the human body supported on the support surface.

In embodiments, the at least one thermoregulating chamber includes a fluid from the group of fluids consisting of: a phase change fluid, carbon dioxide, nitrous oxide, a chemically reactive fluid that cools during a chemical reaction, and a chemically reactive fluid that heats during a chemical reaction.

In embodiments, the at least one thermoregulating chamber includes a chamber circulation pump that circulates the fluid contained therein.

According to still another aspect of the invention, there is provided, a thermoregulating unit for increasing metabolism of at least a portion of a human body, the thermoregulating unit comprising a support surface configured for supporting at least a portion of a human body, at least one cooling coil contained within the support surface, the at least one cooling coil configured to draw heat away from the at least a portion of the human body supported on the support surface.

In embodiments, the one coil passively draws the heat away.

In embodiments, the thermoregulating unit includes at least one passive thermoregulating chamber thermally coupled to the at least one coil, the at least one passive thermoregulating chamber configured to draw heat away from the at least one coil.

In embodiments, the at least one passive thermoregulating chamber is thermally coupled to the body and additionally configured to draw heat away from the at least a portion of the human body supported on the support surface.

In embodiments, the at least one passive thermoregulating chamber includes thermoregulating material, which is from the group of materials consisting of: metal pads, liquid capsules, diamond chips, heat pipes, mega flats, and silicon pads.

In embodiments, the at least one passive thermoregulating chamber includes a fan configured to foster heat exchange between the thermoregulating material and the environment.

In embodiments, the at least one passive thermoregulating chamber contains a fluid configured to draw heat away from the at least one coil.

In embodiments, the at least one passive thermoregulating chamber contains materials that act as heat sinks from the group consisting of: metal pads, liquid capsules, diamond chips, heat pipes, mega flats, and silicon pads.

In embodiments, the at least one passive thermoregulating chamber includes a fan configured to foster heat exchange between the thermoregulating material and the environment.

In embodiments, at least a portion of a surface associated with the at least one passive thermoregulating chamber is embedded with heat conductors from the group of: metal, liquid, diamonds, heat pipes, mega flats, and silicon pads.

In embodiments, at least a portion of the surface associated with the at least one passive thermoregulating chamber is contoured to at least partially curve around at least a portion of a human body.

In embodiments, the thermoregulating unit includes a fan configured to foster heat exchange between the heat conductors and the environment.

In embodiments, the thermoregulating unit includes at least one cooling unit connected to the coils and configured to draw heat away from the coils.

In embodiments, the at least one cooling unit is powered by direct current.

In embodiments, the at least one cooling unit includes at least one sensor that causes the cooling unit to provide greater cooling to warmer parts of the body.

In embodiments, thermoregulating unit is configured for use in a vehicle. In embodiments, the support surface comprises a chamber.

In embodiments, the chamber includes apertures configured to allow passage of a gas contained therein into the surrounding environment in response to movement by at least one of: the vehicle, and the user.

In embodiments, the apertures are configured to allow passage of gas from the surrounding environment into the one chamber.

According to a further aspect of the invention, there is provided a thermoregulating unit for increasing metabolism of at least a portion of a human body, the thermoregulating unit comprising a support surface which comprises an upholstered surface enclosing one chamber, the upholstered surface including thermoregulating material juxtaposed on the support surface, the thermoregulating material configured to contact and cause a first rate of heat loss from at least a portion of a human body supported on the support surface and into the one chamber, an aperture set connecting the one chamber with the environment surrounding the support surface and configured to draw heat out of the one chamber to the environment.

In embodiments, the thermoregulating unit includes one vibrator operatively associated with the unit, the one vibrator configured to cause frictional interaction between the portion of the human body and the support surface, thereby causing vasodilation that causes a second, faster, rate of heat loss from at least a portion of a human body supported on the support surface.

In embodiments, the thermoregulating material is from the list of materials consisting of metal pads, liquid capsules, diamond chips, and silicon pads.

In embodiments, the thermoregulating unit includes a thermoregulating chamber comprising a fluid from the group of fluids consisting of: a phase change fluid, carbon dioxide, nitrous oxide, a chemically reactive fluid that cools during a chemical reaction, and a chemically reactive fluid that heats during a chemical reaction.

According to still a further aspect of the invention, there is provided, a thermoregulating unit for increasing metabolism of at least a portion of a human body, the thermoregulating unit comprising a support surface comprising an upholstered surface enclosing one chamber, the upholstered surface including at least two sets of apertures: a first aperture set comprising body-contacting apertures configured to contact and draw heat into the one chamber from a portion of at least a portion of a human body supported on the support surface, and a second aperture set comprising environmental-contacting apertures configured to draw heat from the one chamber into the surrounding environment.

In embodiments, at least one of the apertures has a diameter of at least between about 1.0 and 6.0 millimeters.

In embodiments, at least one of the apertures has a diameter of no more than between about 7.0 and 15.0 millimeters.

According to a still further aspect of the invention, there is provided a cushion having thermoregulating properties, the cushion comprising an apertured surface configured for contacting a portion of a human body, the apertured surface configured to draw heat away the portion of the human body at a first rate, and one vibrating unit operatively associated with the cushion, the one vibrating unit configured to cause frictional interaction between the portion of the human body, thereby causing vasodilation that draws heat away the portion of the human body at a second, increased, rate.

According to still a further aspect of the invention, there is provided a thermoregulating unit for causing weight loss by increasing metabolism of at least a portion of a human body, the thermoregulating unit comprising a cushion having thermoregulating properties, the cushion comprising an apertured surface configured for contacting a portion of at least a portion of a human body, the apertured surface configured to draw heat away from the portion of the human body at a first rate, and one vibrating unit operatively associated with the cushion, the one vibrating unit configured to cause frictional interaction between the portion of the human body, thereby causing vasodilation that draws heat away from the portion of the human body at a second, increased, rate.

According to still another, additional further aspect of the invention, there is provided, a thermoregulating unit for causing weight loss by increasing metabolism of at least a portion of a human body, the thermoregulating unit comprising a support surface configured for supporting at least a portion of a human body, one elongate tubular portion connected to the support surface, the elongate tubular portion having two ends comprising an inlet end and an outlet end, a cooling fluid contained within the elongate tubular portion, and a pump having an inlet connected to the inlet end and an outlet connected to the outlet end, the pump configured to circulate the cooling fluid through the one elongate tubular portion.

In embodiments, the one elongate tubular portion passively draws heat away from the at least a portion of the human body supported on the support surface.

In embodiments, at least a portion of the elongate tubular portion passes through a cooling unit from According to still an additional aspect of the invention, there is provided a chair that causes weight loss by increasing metabolism in a human body, the group of cooling units comprising: a freezer, a refrigerator, a room air conditioner, a central air conditioner, and an air conditioner in a vehicle.

In embodiments, the support surface base comprises at least one of: one seat, one backrest, one headrest, one armrest, one hand rest, one leg rest, and one footrest.

In embodiments, the one elongate tubular portion is juxtaposed on a portion of at least one of the: one seat, one backrest, one headrest, one armrest, one hand rest, one leg rest, and one footrest.

In embodiments, wherein the support surface comprises at least two of the: one seat, one backrest, one headrest, one armrest, one hand rest, one leg rest, and the one footrest; is configured to form into a sleeping surface.

In embodiments, wherein the support surface comprises at least two of the: one seat, one backrest, one headrest, one armrest, one hand rest, one leg rest, one footrest; are configured as a sleeping surface.

In embodiments, the thermoregulating unit includes a cooling unit connected to the one elongate tubular portion, the cooling unit configured to cool the cooling fluid when the cooling fluid passes therethrough.

In embodiments, the cooling unit comprises at least one of: a Peltier unit, and an evaporative cooling unit.

In embodiments, the cooling unit is juxtaposed on a portion of the at least: one support surface base, one seat, one backrest, one headrest, one armrest, one hand rest, one leg rest, and one footrest.

In embodiments, the cooling unit is powered by a power supply from the group consisting of: a self-contained battery, a car battery, and an alternating current.

In embodiments, the chair comprising one chair component configured to contact at least a portion of a human body, one first thermoregulating unit includes acoustic insulation at least partially surrounding at least one of the: cooling unit, and pump.

In embodiments, the thermoregulating unit includes a noise canceling wave generator operatively associated with at least one of the: cooling unit, and pump.

In embodiments, the thermoregulating unit includes at least one sensor configured to measure one parameter of the at least one body portion from the group of parameters comprising: user weight, body temperature, caloric output, heart rate, and respiration rate.

In embodiments, the thermoregulating unit includes a controller connected to the sensor, the controller adapted to actively control a rate at which the pump circulates the cooling fluid based upon the one parameter.

In embodiments, the controller additionally regulates at least one of: a rate of user heat loss, and a level of user discomfort.

In embodiments, the cooling unit comprises at least one of: a Peltier unit, and an evaporative cooling unit.

In embodiments, the sensor is configured to sense when the user has left the thermoregulating unit.

In embodiments, the controller is configured, when the user has left the thermoregulating unit, to automatically turn off at least one of the: cooling unit, and pump.

In embodiments, the controller is programmed with a seasonally adjusted energy saver mode that automatically conserves energy consumption at peak energy consumption hours by at least one of the: cooling unit, and pump.

In embodiments, the thermoregulating unit includes a heating element configured to raise the temperature of the cooling fluid to regulate the rate of heat loss, or the level of discomfort, or both.

In embodiments, the cooling unit includes a controller configured to control the cooling unit to provide a specific level of cooling without exceeding a chosen tolerable level of discomfort.

In embodiments, the thermoregulating unit includes at least one sensor connected to the controller, the at least one sensor configured to sense the temperature of the at least a portion of the supported human body.

In embodiments, the at least one sensor is configured to cause the cooling unit to cause a calorie loss via the at least a portion of the supported human body.

In embodiments, the thermoregulating unit includes a display configured to display at least one of the: calorie loss of a user, and weight of a user.

In embodiments, the thermoregulating unit includes a speaker system.

In embodiments, the display and the speaker system are configured to provide audiovisual output to the user. In embodiments, the audiovisual output is configured to aid in caloric loss of the user.

In embodiments, the audiovisual output comprises at least one of: upper extremity exercises, lower extremity exercises, and audiovisual patterns associated with breathing by the user.

In embodiments, the display includes an input by which a user programs a temperature provided by the cooling unit.

In embodiments, the input comprises a touch screen. In embodiments, the input comprises a LED.

In embodiments, the display displays two or more temperature patterns provided by the cooling unit and the input allows a user to input one of the two or more temperature patterns.

In embodiments, an internet link is included and said display is located on an internet website and, by inputting a user identification string into said website, the display displays at least one of the: calorie loss of the user; and weight of the user.

In embodiments, the thermoregulating unit includes a central billing station to which the unit is connected, the central billing station configured to bill the user for use of the thermoregulating unit.

In embodiments, the sensor includes a wireless transceiver configured to communicate with a remote wireless controller unit, the remote wireless control unit including a display.

In embodiments, the remote wireless controller unit includes an input by which a user programs a temperature provided by the cooling unit.

In embodiments, the remote wireless controller unit includes an input by which a user chooses a temperature pattern and the display displays two or more temperatures pattern.

In embodiments, the two or more temperatures patterns include a first pattern with increased caloric loss and a second pattern with decreased caloric loss.

In embodiments, the thermoregulating unit includes an identification input configured to identify at least one user upon input of at least one identification string.

In embodiments, the identification input comprises a smartcard reader and the at least one identification string is contained on at least one smartcard.

In embodiments, the thermoregulating unit includes a controller, which, based upon the input of the at least one smartcard, automatically causes at least one of the cooling units to operate at a preset temperature, and the thermoregulating unit to assume a preset configuration.

In embodiments, the at least one smartcard comprises at least two smartcards, each smartcard being input by at least one of at least two users of said dual thermoregulating units.

In embodiments, the thermoregulating unit includes one vibrator operatively associated with the unit, the one vibrator configured to cause frictional interaction between the portion of the human body and the support surface, thereby causing vasodilation that increases a rate of heat loss.

In embodiments, the unit is configured for use in a vehicle.

In embodiments, the one elongate tubular portion includes a foam material.

In embodiments, the one elongate tubular portion includes thermoregulating materials from the group consisting of: metal pads, liquid capsules, diamond chips, heat pipes, mega flats, and silicon pads.

In embodiments, the one elongate tubular portion includes a fan configured to foster heat exchange between the thermoregulating material and the environment.

In embodiments, the one elongate tubular portion includes a fluid from the group of fluids consisting of: a phase change fluid, carbon dioxide, nitrous oxide, a chemically reactive fluid that cools during a chemical reaction, and a chemically reactive fluid that heats during a chemical reaction.

In embodiments, the thermoregulating unit includes at least one inflatable cushion connected to the support surface.

In embodiments, the support surface includes thermoregulating material from the group of materials consisting of: metal pads, liquid capsules, diamond chips, heat pipes, mega flats, and silicon pads.

In embodiments, the support surface is operatively associated with a fan configured to foster heat exchange between the thermoregulating material and the environment.

In embodiments, at least a portion of the support surface is contoured to at least partially curve around the at least a portion of the human body.

In embodiments, the support surface includes apertures configured to allow passage of a gas contained therein into the surrounding environment.

In embodiments, at least one of the apertures has a diameter of at least between about 1.0 and 6.0 millimeters.

In embodiments, at least one of the apertures has a diameter of no more than between about 7.0 and 15.0 millimeters.

In embodiments, the support surface comprises a material that facilitates removal of heat from a body selected from the group of materials consisting of: polyethylene, polyvinyl chloride, polyurethane, nylon, and a biocompatible polymer fiber.

In embodiments, the support surface comprises a material having a thickness of at least about between about 1.0 and 6.0 millimeters.

In embodiments, the support surface comprises a material having a thickness of no more than between about 7.0 and 15.0 millimeters.

In embodiments, the support surface comprises a material that facilitates removal of heat from a body selected from the group of materials consisting of: nitinol, stainless steel shape memory materials, metals, synthetic biostable polymer, a natural polymer, and an inorganic material.

In embodiments, the biostable polymer comprises a material from the group of materials consisting of: a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, a polyester, an aromatic polyester, a polysulfone, and a silicone rubber.

In embodiments, the natural polymer comprises a material from the group of materials consisting of a polyolefin, a polyurethane, a Mylar, a silicone, and a fluorinated polyolefin.

In embodiments, the support surface comprises a material having a property from the group of properties consisting of: compliant, flexible, plastic, and rigid.

In embodiments, the support surface comprises an API that biologically aids in increasing metabolism of a user when the user is nude or wearing garments configured to absorb the API.

In embodiments, the API comprises a chemotherapeutic selected from the group consisting of: peptides, proteins, calcitonin, analgesics, antidepressants, antihistamines, anti-inflammatory agents, antiirritants, antilipemics, antipruritics, non-steroidal anti-inflammatory agents, vasodilators, and mixtures thereof.

In embodiments, the API comprises an analgesic selected from the group consisting of benzocaine, butamben picrate, dibucaine, dimethisoquin, dyclonine, lidocaine, pramoxine, tetracaine, salicylates and derivatives, esters, salts, and mixtures thereof.

In embodiments, the material is formed by a process from the group of processes consisting of: knitting, braiding, knotting, wrapping, interlacing, and electrospinning.

Dual thermoregulating units for increasing metabolism of a human body, the dual thermoregulating units comprising a support surface configured for supporting at least a portion of a human body, one first thermoregulating unit juxtaposed on the support surface and configured to produce a first temperature, one second thermoregulating unit juxtaposed on the support surface and configured to produce a second temperature, and one temperature controller connected to the one first and the one second thermoregulating units, the one temperature controller configured to vary the first produced temperature and vary the second produced temperature such that the first produced temperature is varied independently of the second produced temperature.

In embodiments, the one first thermoregulating unit and the one second thermoregulating unit are unremovably affixed to a chair.

In embodiments, the one first thermoregulating unit and the one second thermoregulating unit are removably affixed to a chair.

In embodiments, the controller is additionally connected to one first sensor configured to measure a metabolic-based parameter of at least one first portion of the human body, and one second sensor configured to measure the metabolic-based parameter of at least one second portion of the human body.

In embodiments, the metabolic-based parameter is from the group of parameters comprising: temperature, weight, caloric change, heart rate, and respiration rate.

In embodiments, the controller is configured to calculate caloric loss based upon the measurement of the metabolic-based parameter of the at least one first portion of the human body, and the at least one second portion of the human body.

In embodiments, the controller is configured to vary the temperature produced by the one first thermoregulating unit and the one second thermoregulating unit based upon the measurement of the metabolic-based parameter.

In embodiments, the controller is configured to ensure a chosen tolerable level of discomfort by the one first thermoregulating unit, and the one second thermoregulating unit.

In embodiments, the produced temperature of the one first thermoregulating unit is varied above an ambient temperature of the at least one first portion of the human body, and the one second thermoregulating unit is varied below an ambient temperature of the at least one second portion of the human body.

In embodiments, the produced temperature of the one first thermoregulating unit is varied above an ambient temperature of the at least one first portion of the human body, and the one second thermoregulating unit is varied above an ambient temperature of the at least one second portion of the human body.

In embodiments, the produced temperature of the one first thermoregulating unit is varied below an ambient temperature of the at least one first portion of the human body, and the one second thermoregulating unit is varied below an ambient temperature of the at least one second portion of the human body.

In embodiments, the dual thermoregulating units comprise one chair component comprises a seat support surface configured to support at least a portion of the supported contacted portion of the human body.

In embodiments, the dual thermoregulating units additionally comprises at least one second component comprising at least one of: one a seat and, one backrest, one headrest, one armrest, one hand rest, one leg rest, and one footrest.

In embodiments, the at least two components are positionally adjustable.

In embodiments, the at least two components are positionally adjustable to provide one first position during a first period, and one second position during a second period.

In embodiments, the one first position is adjusted prior to contacting at least a portion of the human body, and the one second position is adjusted following contacting at least a portion of the human body.

In embodiments, the one first position is adjusted prior to operation of the one first thermoregulating unit and the one second thermoregulating unit, and the one second position is adjusted during operation of the one first thermoregulating unit and the one second thermoregulating unit.

In embodiments, the one first position is in a first direction, and the one second position is in a second direction.

In embodiments, the positional adjustment is configured to vary the metabolic-based parameter.

In embodiments, the positional adjustment comprises at least one of a: messaging motion, and a vibrating motion.

In embodiments, the dual thermoregulating units additionally include a housing located below the seat, the housing configured to house at least a portion of the one first thermoregulating unit, and the one second thermoregulating unit.

In embodiments, at least a portion of one chair component is embedded with heat conductors from the list comprising metal, liquid, diamonds, heat pips, mega flats, and silicon pads.

In embodiments, at least a portion of the group of materials consisting of: metal pads, liquid capsules, diamond chips, heat pipes, mega flats, and silicon pads.

In embodiments, at least one of the dual thermoregulating units includes a fan configured to foster heat exchange between the thermoregulating material and the environment.

In embodiments, at least one of the dual thermoregulating units includes a fluid from the group of fluids consisting of: a phase change fluid, carbon dioxide, nitrous oxide, a chemically reactive fluid that cools during a chemical reaction, and a chemically reactive fluid that heats during a chemical reaction.

In embodiments, at least one of the dual thermoregulating units comprises a material that facilitates removal of heat from a body selected from the group of materials consisting of: polyethylene, polyvinyl chloride, polyurethane, nylon, and a biocompatible polymer fiber.

In embodiments, the material has a thickness of at least about between about 1.0 and 6.0 millimeters.

In embodiments, the material has a thickness of no more than between about 7.0 and 15.0 millimeters.

In embodiments, the material comprises a material that facilitates removal of heat from a body selected from the group of materials consisting of: nitinol, stainless steel shape memory materials, metals, synthetic biostable polymer, a natural polymer, and an inorganic material.

In embodiments, the biostable polymer comprises a material from the group of materials consisting of: a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, a polyester, an aromatic polyester, a polysulfone, and a silicone rubber.

In embodiments, the natural polymer comprises a material from the group of materials consisting of: a polyolefin, a polyurethane, a Mylar, a silicone, and a fluorinated polyolefin.

In embodiments, the material comprises a material having a property from the group of properties consisting of: compliant, flexible, plastic, and rigid.

In embodiments, the material includes an API that biologically aids in increasing metabolism of a user when the user is nude or wearing garments configured to absorb the API.

In embodiments, the API comprises a chemotherapeutic selected from the group consisting of: peptides, proteins, calcitonin, analgesics, antidepressants, antihistamines, anti-inflammatory agents, antiirritants, antilipemics, antipruritics, non-steroidal anti-inflammatory agents, vasodilators, and mixtures thereof.

In embodiments, the API comprises an analgesic selected from the group consisting of: benzocaine, butamben picrate, dibucaine, dimethisoquin, dyclonine, lidocaine, pramoxine, tetracaine, salicylates and derivatives, esters, salts, and mixtures thereof.

In embodiments, the material is formed by a process from the group of processes consisting of: knitting, braiding, knotting, wrapping, interlacing, and electrospinning.

In embodiments, at least a portion of at least one of the dual thermoregulating units is embedded with heat conductors from the group comprising: metal, liquid, diamonds, heat pipes, mega flats, and silicon pads.

In embodiments, at least a portion of at least one of the dual thermoregulating units component is contoured to at least partially curve around at least a portion of a human body.

In embodiments, the one first sensor and the one second sensor are configured to sense when the user has left the dual thermoregulating units.

In embodiments, the controller is configured, when the user has left the dual thermoregulating units, to automatically turn off at least one of the: one first thermoregulating unit, and one second thermoregulating unit.

In embodiments, the controller is programmed with a seasonally adjusted energy saver mode that automatically conserves energy consumption at peak energy consumption hours of at least one of the: one first thermoregulating unit, and one second thermoregulating unit.

In embodiments, the dual thermoregulating units include a display configured to display at least one of the: calorie loss of a user, and weight of a user.

In embodiments, the dual thermoregulating units include a speaker system.

In embodiments, the display and the speaker system are configured to provide audiovisual output to the user.

In embodiments, the audiovisual output is configured to aid in caloric loss of the user.

In embodiments, the display includes an input by which a user changes at least one of a: position, and temperature of at least one of the dual thermoregulating units. In embodiments, the input comprises a touch screen. In embodiments, the input comprises a LED.

In embodiments, the dual thermoregulating units include an internet link and the display is located on an internet website and, by inputting a user identification string into said website, the display displays at least one of the: calorie loss of the user; and weight of the user.

In embodiments, the dual thermoregulating units include a central billing station to which the unit is connected, the central billing station configured to bill the user for use of the dual thermoregulating units.

In embodiments, the sensor includes a wireless transceiver configured to communicate with a remote wireless controller unit, the remote wireless control unit including a display.

In embodiments, the remote wireless controller unit includes an input by which a user inputs at least one of a: temperature, and motion.

In embodiments, the input includes an identification input configured to identify at least one user upon input of at least one identification string.

In embodiments, the identification input comprises a smartcard reader and the at least one identification string is contained on at least one smartcard.

In embodiments, the controller, based upon the input of the at least one smartcard, automatically causes at least one of the dual thermoregulating units to operate at a preset temperature, and assume a preset configuration.

In embodiments, the at least one smartcard input comprises at least two smartcards, each smartcard being input by at least one of at least two users of said dual thermoregulating units.

The present invention successfully addresses the shortcomings of the presently known configurations by providing thermoregulating units configured to increase body metabolism.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the thermoregulation science.

### BRIEF DESCRIPTION OF THE DRAWINGS

Some embodiments of the invention of a thermoregulating unit are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a thermoregulating unit, according to embodiments of the present invention;

FIGS. 2 and 3 show alternative designs of the thermoregulating unit of FIG. 1, according to embodiments of the present invention.

FIGS. 4A-4C show alternative designs of the thermoregulating unit of FIG. 1 that include wheels, according to embodiments of the present invention;

FIGS. 5A and 5B show designs of heat pipe thermoregulating units, according to embodiments of the present invention;

FIG. 5C shows a thermoregulating unit equipped with the heat pipe thermoregulating units of FIG. 5B, according to embodiments of the present invention; and

FIG. 5D shows a heat flow pattern through heat pipe thermoregulating units of FIG. 5B and a close up of a portion of the thermoregulating seat and pump of FIG. 1, according to embodiments of the present invention.

### DESCRIPTION OF EXEMPLARY EMBODIMENTS

The present invention, in some embodiments thereof, is of thermoregulating units that increase body metabolism through temperature reduction.

The principles and operation of thermoregulating units having passive thermoregulation chambers, according to some embodiments of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Passive Thermoregulation Unit

Referring now to the drawings:

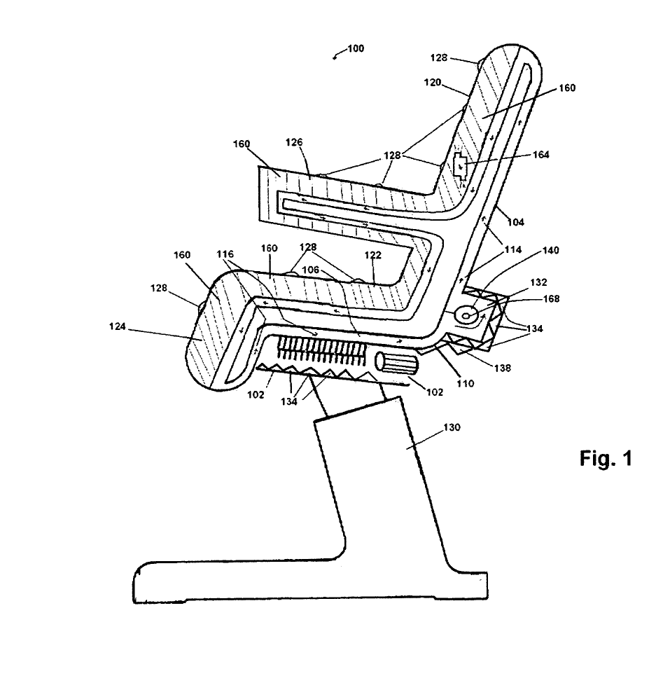

FIG. 1 illustrates a chair configured as a thermoregulating unit 100 including a passive thermoregulating chamber 160 configured to passively draw heat away from the body of a user sitting on thermoregulating unit 100; causing the body to increase metabolic output to facilitate weight loss.

As used herein, the word “passively” with respect to heat loss, heat exchange and/or heat sinks refers to any material that absorbs, retains and/or exchanges heat without active input of energy during the heat loss, and/or exchange.

In embodiments, thermoregulating chamber 160 includes materials that are heat conductors that passively take heat away from the body of the user sitting in thermoregulating unit 100 and release the heat passively to the environment.

For example, in non-limiting embodiments, thermoregulating chamber 160 includes an open cell foam material that acts as a passive reservoir of heat. Open cell foam absorbs and releases heat, from the body temperature of a user, for example as the user shifts weight on the foam and draws in cooler environmental air while releasing hotter air from within the foam.

Additionally or alternatively, the surface of thermoregulating chamber 160 and/or the interior of thermoregulating chamber 160 includes metal pads, liquid capsules, diamond chips, and/or silicon pads that act as heat sinks. In further embodiments, described below, heat sinks configured in longitudinal columns extending between the user and the environment, passively exchange heat with the environment.

In embodiments, the foam material has a thickness of at least between about 1 and 10 centimeters. In alternative embodiments, the material has a thickness of no more than between about 11 and 30 centimeters. Additionally, the heat sinks noted above can be elastic in nature to provide comfort to the user supported by thermoregulating chamber 160.

In still further embodiments, thermoregulating chamber 160 comprises a space containing a fluid, comprising a liquid or gas that aids in removing heat from the body of a user sitting on thermoregulating unit 100 by acting as a passive heat reservoir that takes in heat at the contact surface with a user and passes the heat out through surfaces that are substantially in contact with the environment, for example the air surrounding chamber 160.

In non-limiting examples, the fluid in thermoregulating chamber 160 comprises air, a phase change liquid, a chemically reactive fluid that cools and/or heats during a chemical reaction, carbon dioxide, and/or nitrous oxide.

In embodiments presented herein, thermoregulating unit 100 is configured to promote a temperature that is below the body temperature of the user to cause the user body to resort to various physiological mechanisms to maintain body temperature, thereby increasing metabolic output.

In further embodiments, thermoregulating unit 100 optionally includes mechanisms described below, configured to produce significantly lower temperatures in the user body such that the user body shivers, causing the above-noted 2.5 times increase in metabolic rate; thereby facilitating weight loss. Optionally, the user's base metabolism goes up by at least 10%, or at least 20%, or at least 40%, and the increase lasts for at least one day, at least one week, or at least one month.

In other embodiments, thermoregulating unit 100 influences the user base metabolism during the time in which the user is in contact with unit 100. Further, thermoregulating unit 100 is configured with materials that do not change the base metabolism to create discomfort, for example, promoting a change in the base metabolism rate by less than 10% or even less than 2%.

Internal Chamber Pump

In embodiments, thermoregulating chamber 160 includes a chamber circulation pump 164 that circulates the fluid contained therein, thereby exchanging fluid that has been warmed by the body of the user, with cooler fluid in contact with the cooler surrounding environment, to pass heat away from the body of the user.

In embodiments, chamber pump 164 circulates chemicals that clean the circulating fluid, and remove odors that may otherwise occur during extended service. Optionally chamber pump 164 includes a drain (not shown) that allows a user to periodically change fluid and/or the cleaning chemicals.

External Chamber Pump

Alternatively or additionally, thermoregulating unit 100 includes an external pump 132 that is external to thermoregulating chamber 160. External pump 132 circulates fluid such as water, another cooling fluid, or a gas, through thermoregulating unit 100 via an inlet tube 104 in a forward direction 114.

While passing through thermoregulating unit 100, the fluid is heated by the body and/or the heat sinks, and returns by way of an outlet tube 106 in a direction 116 to a cooling unit 102 where the fluid is cooled down again. In this manner, heat from the fluid contained in thermoregulating chamber 160 is vented away from the user.

In further embodiments noted below, embodiments of thermoregulating unit 100 utilize ice packs and/or phase change liquids to regulate temperature of a user.

Cooling Unit

In further embodiments, thermoregulating unit 100 includes cooling unit 102 that cools fluid passing through pump 132.

Cooled fluid passes through a backrest 120, an armrest 126, a seat 122 and a footrest 124, thereby drawing off heat from chambers 160, and maintaining cooling of a person sitting on thermoregulating unit 100 for extended periods. In embodiments, thermoregulating unit 100 optionally includes the following components not shown: a backrest, headrest, a hand rest and a footrest through which cooled fluid passes.

Optionally, backrest 120, armrest 126, seat 122 and footrest 124 optionally contain cooling units 102 and are configured to be positioned by the user to form a flat bed suitable for sleeping.

In such embodiments, during sleep the user continues to enjoy metabolic regulation that fosters weight loss due to the heat exchange with cooling unit. In alternative embodiments thermoregulation unit 100 is configured as a flat sofa so that the user has the option to sit or lie down, for example while watching television, or go to sleep with a blanket and pillow.

Alternatively, thermoregulating unit 100 comprises only seat 122 as a single large mattress for sleeping.

Cooling unit 102 optionally uses a solid-state cooling method such as the Peltier method. Alternatively, cooling unit 102 comprises an evaporative cooling method.

In further embodiments, cooling unit 102 comprises a “Karno machine” system used in air conditioners whereby a compressor compresses the fluid, which then is transported to expand upon contact with a warm area, thereby cooling the area. Alternatively, cooling unit uses absorption methods of heat exchange, for example in a heat exchange using lithium bromide.

In still alternative embodiments, cooling unit 102 comprises a closed piston heat-input cooler, as exemplified in a Stirling engine.

The many methods for cooling unit to provide effective cooling, wherein cooling unit provides a specific level of heat exchange that fosters a known level of heat loss, are well known to those familiar with the art.

As will be explained below, some embodiments of the invention include inserts that are cooled, for example in a refrigerator, and used until losing their cooling influence on the user, after which the inserts are returned to the refrigerator.

Parameter Measurements

In embodiments, thermoregulating unit 100 includes sensors 128 that measure the temperature of liquid within thermoregulating chambers 160 and a controller 110 regulates the flow of fluid through cooling unit 102 and/or external pump 132.

For example, controller 110 may turn cooling unit 102 and/or external pump 132 on or off to regulate the temperature of the fluid. Optionally controller 110 additionally controls the rate of pumping of fluid by external pump 132 and/or circulation pump 164, thereby maintaining a comfortable temperature for the user. At a faster pumping rate through cooling chamber 102, for example, pump maintains a low temperature in fluid due to the fast rate of heat exchange that the increased flow through cooling unit 102 engenders. At a slower pumping rate, the fluid is in contact with the user for longer periods of time, thereby warming, and maintaining a less cooling temperature.

Alternatively or additionally, sensors 128 measure parameters from the subject legs, thighs, buttocks, back and/or arms where the body contacts thermoregulating unit 100. In the event that sensors 128 sense absence of blood flow, indicating vasoconstriction; a sharp drop in temperature in a body area of the subject and/or extended periods of shivering in the user, controller 110 automatically causes cooling unit 102 and/or pump 132 to slow down or stop functioning.

As will be explained below, in embodiments that include transducers that adjust the position of the components of thermoregulating unit 100, controller optionally adjusts position to increase and/or decrease contact between unit 100 and the user, thereby further influencing thermoregulation.

In some embodiments, controller 110 can be set with time scales and moderate sensing parameters, such that when sensors 128 sense a small drop in temperature, even for a relatively short period of time, controller automatically causes cooling unit 102 and/or pump 132 to compensate and bring the user up to a predetermined and/or prearranged temperature.

Sensors 128 provide feedback to controller 110 so that thermoregulating unit 100 operates in a manner wherein blood flow of the user is not reduced, for example when sensors 128 sense vasoconstriction in a body part of the user.

In further embodiments, one or more of sensors 128 are pressure sensitive and automatically sense the presence of a seated person and as a result, cooling unit 102 immediately and automatically turns on in response to a user sitting on thermoregulating unit 100.

While cooling unit 102 is shown located below seat 122, cooling unit 102 is optionally located behind backrest 120, armrests 126 and/or in footrest 124. Optionally multiple cooling units 102 may be located in each of the above components and optionally each have their own user activated switch. In this manner, multiple cooling units 102 will provide a variety of levels of cooling, and in different components, based upon user preference.

In still further embodiments, cooling unit 102 is located within a seat support post 130, as will be explained below.

Heating Unit

In further embodiments, cooling unit 102 incorporates a heating unit 168 to allow rapid adjustment of temperature of the fluid passing through thermoregulating unit 100 for user comfort and/or producing cycles of cooling followed by heat in order to maintain vasodilation of the subject to facilitate heat loss, thereby facilitating weight loss.

As will be explained below, heating unit 168 may not only be used for controlling the temperature of the contact fluid, but for heating up specific regions of the user body, while other regions are being cooled, and/or for maintaining vasodilation that facilitates heat loss.

While heating unit 168 is shown as part of pump mechanism 140, heating unit 168 may be integrated into cooling unit 102, for example comprising a heating coil within a chamber within cooling unit that functions when cooling unit 102 is not actively cooling the passing fluid.

While armrests 126, seat 122 and/or footrest 124 are shown as continuous structures, other designs are contemplated. For example, armrests 126, seat 122 and/or footrest 124 may comprise individual coils of tubing, each connected to a separate external pump 132 and/or cooling unit 102.

Acoustic Properties

Similar to a compressor assembly on a refrigerator, cooling units 102 and pumps 132 may be associated with rumblings and/or vibrations upon starting and stopping.

While a person is often not aware of such noises generated by the refrigerator, a person sitting in thermoregulating unit 100 would be much more aware of such noises. Further, cooling units 102 and pumps 132 may cycle through several cycles in a hot environment within a short period, for example a half hour, disrupting user quiet at an intolerable frequency.

To reduce the noise level of thermoregulating unit 100, external pump 132 and cooling unit 102 are optionally enclosed in an acoustic insulator 134 that suppresses noise associated with operation.

In further embodiments, noise-canceling waves are optionally generated by a sound wave generator 138 and cancel sound waves at specific decibel and pitch that can be irritating to a user.

In still further embodiments, sound wave generator 138 is configured to cancel a wide range of pitches that are associated, for example with ambient noise levels such as passing traffic. In such configurations, sound wave generator 138 can provide a comforting island in a sea of noise in addition to providing metabolic regulation through heat loss.

In addition to the noise dampening options noted above, to reduce the effect of vibrations caused by starting and stopping, external pump 132 and/or cooling unit 102 optionally include vibration-dampening suspensions, for example springs and/or shock absorbers between external pump 132 and/or cooling unit 102 and a supporting surface (not shown).

Alternative Pump Mechanisms

In embodiments, external pump 132 includes a pump mechanism 140 comprising a rotor that circulates the liquid, while in other embodiments, pump mechanism 140 comprises an accordion-like suction pump. In still other embodiments, pump mechanism comprises an air pump and circulates air through inlet tube 104 and outlet tube 106; the many configurations of pump mechanism 140 being well known to those familiar with the art.

Positional Adjustments

Optionally, the height and/or angles formed by armrests 126, seat 122, backrest 120, and/or footrest 124, of thermoregulating unit 100 are optionally adjustable to increase the comfort of the subject. Optionally, in response to a touch by the user, controller 110 automatically adjusts thermoregulating unit 100 to an initial height and position to aid the user in accessing thermoregulating unit 100.

Such mechanical adjustments are especially helpful to the obese who may be challenged in simply getting into a chair or couch, let alone raising their feet onto footrest 124.

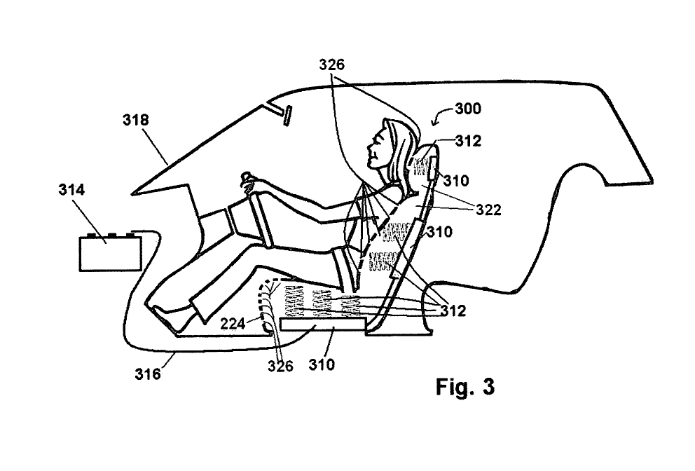

Sensors connected to transducers that adjust the position of armrests 126, seat 122, backrest 120, and/or footrest 124 are well known in the art. Further, motors 490, 492, 494 and 496 that adjust the position of thermoregulating unit 400 (FIG. 4A), are described below.

In addition to providing positional adjustments, thermoregulating unit 100 can, after the user is seated, be brought to an optimal position for ensuring thermoregulation. For example, by activating a control that brings armrests 126 to a height that is more supportive of the user arms, the surface area of the arms of the user will have optimal contact with armrests 126, thereby efficiently increasing thermoregulation.

Alternatively, controller 110 optionally directs seat support post 130 to raise the user such that the user legs are above the floor, allowing full contact between the user lower extremities and leg rest 124, thereby facilitating greater thermoregulation.

In addition, sensors may be used to detect sleeping patterns of the user. Sleep detecting sensors have application when a user is sensitive to cooling while awake, but not so when asleep.

In such cases, sensors that detect movement of the user may be triggered when the user stretches out into a sleeping position, and lies still. When the sensors detect that the user is awake, thermoregulatory unit 100 is kept at a higher temperature. When the sensors detect that the user is sleeping, thermoregulatory unit 100 is switched to a lower temperature.

Heat Exchange Blocks

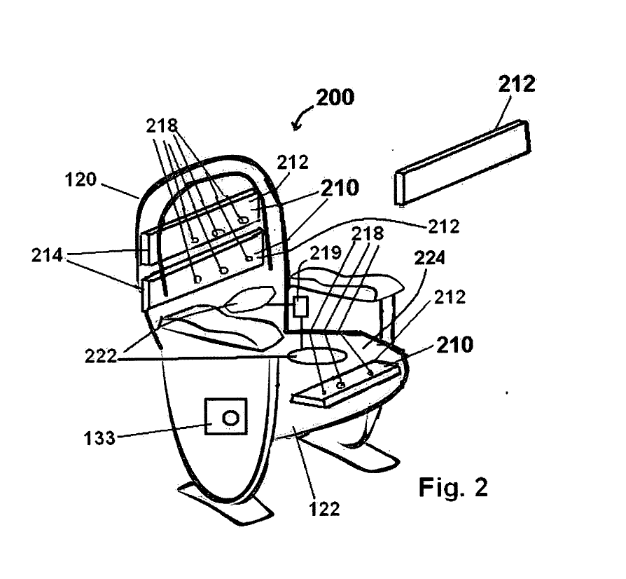

FIG. 2 shows a chair configured as a block heat exchange thermoregulating unit 200, including insertable heat exchange blocks 212 that cool thermoregulating unit 200.

While heat exchange blocks 212 are shown as having a rectangular shape, many shapes are optionally contemplated, including: elongate cylindrical rods, spheres, and polygons of virtually any shape.

In embodiments, insertable heat exchange blocks 212 contain phase-change material and are cooled, for example in a refrigerator for example in the kitchen, or a portable unit located near thermoregulating unit 200.

As shown, heat exchange blocks optionally snap into openings 214 in thermoregulating unit 200 until spanning a passage 210.

Optionally, heat exchange blocks 212 are slid into openings 214 in chair. Alternatively, thermoregulating unit 200 includes drawers that pull in or out of unit 200 (not shown). In such configurations, the user pulls the drawer out of thermoregulating unit 200, inserts heat exchange block 212 into the drawer and pushes the drawer into thermoregulating unit 200.

In some embodiments, the drawers and/or heat exchange blocks 212 include indicators that indicate when cooling blocks need to be replaced, for example based upon the heat of blocks 212. Alternatively, the drawer include sensors that measure time since blocks were installed in unit 200.

In some embodiments, heat exchange blocks 212 contain a chemically reactive fluid and/or material that cools and/or heats during a chemical reaction; requiring recharging by chemical and/or thermal recharging after warming.

Optionally, heat exchange blocks 212 are capable of being cooled in a refrigerating unit or frozen in a freezer multiple times. Alternatively, heat exchange blocks 212 comprise low cost cooling fluids, for example water, that are contained within a low cost plastic bag so that blocks can be thrown out after even one use; an advantage when thermoregulating unit 200 is located in a vehicle, as explained below.

Cushion and Massage Options

Optionally, thermoregulating unit 200 includes one or more inflatable cushions 222, for example a lumbar support inflatable cushion 222 in backrest 120, thereby allowing the subject to customize the comfort provided by thermoregulating unit 200.

In further embodiments, cooling unit includes a massage feature, for example, wherein inflatable cushions 222 are connected to a fluid pump 219 that first inflates and deflates cushions 222 associated with backrest 120 and seat 122, thereby providing a massage to the user.

Optionally, the massage system can be provided independent of thermoregulating unit 200. In still further options, inflatable cushions 222 are inflated with cooling fluid so that the user is cooled during the massage.

In still further embodiments, cushion 222 associated with backrest 120 comprises multiple chambers, for example four or even eight chambers, that are alternatively inflated and deflated with fluid by fluid pump 219, thereby providing a more effective massage of the user's back.

In alternative embodiments, cushions 222 are configured from materials that provide insulation and are located directly over cooling blocks 212 and, by inflating cushions 222. In such embodiments, the subject may individually adjust the contact between body parts and cooling blocks located in seat 122, footrest 124, armrests 126 and/or backrest 120.

Vibration Component

In still further embodiments, thermoregulating unit 200 optionally incorporates a vibrator 133 that serves to stimulate the skin of the subject and optionally provides further metabolic stimulation facilitating weight loss.

In still further embodiments, vibrator 133 causes vasodilation that improves the efficiency of heat loss in the user.

Coiled Thermoregulators

FIG. 3 shows a seat configured with a coiled thermoregulating unit 300 configured for use in a vehicle 318, comprising elongate coils 312 projecting through thermoregulating unit 300. In some embodiments, cooling coils 312 comprise materials that remove heat passively from the user.

Coils 312 are shown in a vehicle seat and not only server to remove heat passively from the user, but provide bounce within unit 300 so that coil environmental air continually passes through apertures 224 and cools coils 312.

During normal driving, coils 312 move with the sway of the vehicle, providing continued movement of air through apertures 326.

In still further embodiments, coils 312 comprise hollow columns of rubberized material that both support the user and remove heat from the user. In embodiments, cooling coils 312 are connected to cooling units 310 that cool coils 312. Cooling units 310 are optionally powered by direct current and are connected via a wire 316 to a car battery 314. Alternatively, cooling units 310 have self-contained batteries.

In further embodiments, the position of cooling units 310 and cooling coils 312 is altered, for example with cooling units 310 against upholstery 224 to provide direct and active cooling to the occupant of the vehicle. Coils 312, optionally extend outward from cooling units 310 in order to act as heat sinks for example when cooling units 310 are solid state.

Alternatively, cooling units 310 are suspended between a first set of coils 312 extending in a first direction and a second set of coils extending in an opposite direction therefrom. Such a configuration is useful when cooling units 310 comprise a thermoregulator having a warm side and a cool side.

Coils 312 connected to the warm side of cooling unit are optionally connected away from the user toward the environment associated with the back and floor-supporting unit 310. At the same time, coils 312 extending toward upholstery 224 bring coolness to the occupant of vehicle 318.

In embodiments, upholstery 224 comprises a material that is configured, in conjunction with apertures 326, to facilitate removal of heat from the occupant of unit 300. Such materials optionally include polyethylene, polyvinyl chloride, polyurethane, nylon, and a biocompatible polymer fiber.

In embodiments, upholstery 224 has a thickness of at least about between about 1.0 and 6.0 millimeters so that air passing through apertures 326 vents more heat during passage of the air.

In alternative embodiments, upholstery 224 has a thickness of no more than between about 1.5 and 7.0 millimeters, for example when cooling units 310 are located next to upholstery 224, so as not to provide unwanted insulation against cooling units 310.

Varying the diameter and density of apertures 326 can also aid in thermoregulation. When apertures 326 are densely spaced greater movement of air through unit 300 occurs, bringing cool air from cooling units 310 up through the upholstery and venting warm air away from the user.

In other instances, when apertures 326 are spaced apart, less venting of air from the environment occurs and may be desirable when cooling units 310 are located near upholstery 224.

In embodiments, upholstery 224 and/or coils 312 comprise a material that facilitates removal of heat from a body selected from the group consisting of: nitinol, stainless steel shape memory materials, metals, synthetic biostable polymer, a natural polymer, and an inorganic material.

In embodiments, upholstery 224 comprises a material having a property from the group of properties consisting of: compliant, flexible, plastic, and rigid. More compliant and flexible materials may be advantageous in ensuring flexibility of upholstery 224 with coils 312 alone, so that during movement of the user and while vehicle 318 is in motion there is greater circulation of air through apertures 326.

Materials that are more rigid may be advantageous in conjunction with cooling units 310 located next to upholstery 224 to ensure more efficient transfer of cooling effects to the occupant.

In embodiments, thermoregulating unit 300 includes fluids, noted above, contained in sealed cylinders that enclose coils 312 and aid in venting heat from coils 312.

Therapy Unit Programming

FIG. 4A shows a chair comprising a movement therapy unit 400 having multiple thermoregulating units 410, 412, 414, and 416, connected to multiple fluid movement tubes 420, 422, 424, 426, respectively.

Wireless controller is optionally supplied with movement therapy unit 400 that wirelessly programs micro processing chips in thermoregulating units 410, 412, 414, and 416.

Wireless unit 440 allows the user to wirelessly adjust the temperature output, duration of output, and temperature cycling of each of thermoregulating units 410, 412, 414, and 416.

In alternative embodiments, wireless unit 440 comprises, a cell phone, PDA, and/or a blueberry unit.

As noted below, any of the thermoregulatory units described herein may include wireless unit 440 that aids in programming thermoregulation, or may include mechanical inputs such as switches and/or dials that adjust thermoregulation, for example temperature cycling of each of thermoregulating units 410, 412, 414, and 416.

Movement Therapy

In embodiments, temperature cycling of thermoregulating units 410, 412, 414, and 416 cause the user to move periodically and/or change the position of upper torso and lower extremities; with the movement causing expenditure of calories in addition to loss of heat due to thermoregulation described above.

In an exemplary embodiment, first back thermoregulating unit 410 pumps higher temperature fluid through fluid movement tube 420 while second back thermoregulating unit 412 pumps lower temperature fluid through fluid movement tube 422; thereby causing the user to assume a first position in unit 400.

Following a period of time, the temperatures are rearranged so that the user assumes another position, thereby possibly causing a caloric loss to the user that aids in weight loss.

In further embodiments, the rate of temperature change is set to be random so that the user continually changes position.

In an example of random temperature change, first back thermoregulating unit 410 causes the temperature of fluid in fluid movement tube 420 to increase at a first rate of increase for a first period.

During this first period, second back thermoregulating unit 412 causes the temperature of fluid in fluid movement tube 422 to decrease at a second rate of decrease for a second period of time, thereby causing the response in the user to randomly and periodically change position to accommodate the changing temperatures.

Optionally, movement therapy unit 400 includes sensors 430, 432, 434, and 436 that are connected to thermoregulating units 410, 412, 414 and 416, respectively. Sensors 430, 432, 434, and 436 sense user parameters including, one or more of: movement of the user, user shivering, and body temperature; with such parameters being used by a controller to calculate the caloric loss of the user.

In embodiments, wireless unit 440 optionally includes a display 442 that displays caloric loss and/or user weight and the user has the option to reprogram the heat or cold cycling of thermoregulating units 410, 412, 414, and 416 through wireless unit 440.

In embodiments, thermoregulating units 410, 412, 414, and 416 include fail-safe circuitry, for example thermister circuits, which prevent fluid in fluid movement tubes 420, 422, 424, and 426 from reaching a temperature that is either above or below a safe level for the user, so that the continually changing temperatures do not negatively impact the user.

In embodiments, prior to using movement therapy unit 400, information regarding patient health and personal history are input into wireless unit 440 and a recommended thermoregulation pattern is automatically programmed wirelessly into thermoregulating units 410, 412, 414, and 416.

Alternatively, a user programs thermoregulating units 410, 412, 414, and 416 manually, for example using mechanical inputs noted below.

In embodiments, display 482 displays a range of recommended thermoregulation patterns, for example a first pattern with increased caloric loss; and a second pattern with decreased caloric loss; and the user has the option to choose between the first and second thermoregulation patterns.

In further embodiments, movement therapy unit 400 is used by multiple users, each with different thermoregulation patterns that are recorded upon a personal smartcard distributed to each user. prior to sitting on movement therapy unit 400, the user passes the personal smartcard past a smartcard reader 480 on wireless unit 440 and each of thermoregulating units 410, 412, 414, and 416 are thereby programmed according to the thermoregulation pattern of the specific user. Additionally, as noted above, thermoregulating unit 400 assumes different positions automatically based upon the information contained on the user smartcard.

In further embodiments, movement therapy unit 400 includes position adjusting motors 490, 492, 494, and 496 that adjust the height of seat 426, and/or the tilt of backrest 422. In this manner, after the user sits in therapy unit 400, it can be adjusted to regulate thermoregulation.

As noted above, such positional changes can influence thermoregulation. For example, as noted above, the height of seat 426 may be increased until the user feet are no longer resting on the ground and the user thighs are totally supported by seat 426, thereby potentially increasing thermoregulation by unit 400.

Additionally, by automatically reclining backrest 422, the weight of the user upper torso is caused to rest more fully against backrest 422, again potentially resulting in increased thermoregulation by unit 400.

Alternatively, the smart card reader comprises a computer keyboard upon which the user types an identifier so that thermoregulating unit 400 assumes the above-noted position.

During a first session, when the thermoregulation pattern changes, whether by user choice or through input of sensors 430, 432, 434 and 436; at a subsequent session, upon passing the personal smartcard past smartcard reader 480, wireless unit 440 optionally automatically programs the changed thermoregulation pattern into thermoregulating units 410, 412, 414, and 416.

In embodiments, movement therapy unit 400 is connected to a computer 482 that has a display 484 upon which a user optionally enters information and which records the user caloric loss during a given session based upon the above-noted parameters including user weight and body temperature.

In further embodiments, computer 482 is connected to the Internet and the user, a therapist and/or a caregiver optionally access a website dedicated to movement therapy unit 400. In such configurations, by entering a user identification code, information about the user and/or a given session is displayed on a display.

In still further embodiments, computer 482 is connected to a central billing station and the amount of time a given user spends on movement therapy unit 400 is recorded. Based upon the user usage time, a bill is generated and sent to the user, health care provider, and/or a third party payer.

In further embodiments, based upon user usage time, discounts can be automatically provided to the user if the user uses unit 400 for a sufficient amount of time during a given period; for example everyday for a month.

Refrigerated Unit

FIG. 4B shows a chair comprising a refrigerated unit 500 including a pump unit 512 that is set in a separate cooling unit 514, for example a freezer or refrigerator. Pump unit 512 is connected to electricity via a plug 528 and pumps fluid through fluid circulation tubes 511 in a chair. Pump unit 512 includes, for example, a fluid temperature sensor 515 that slows, reverses, stops or speeds up the rate at which pump unit 512 pumps fluid through fluid circulation tubes 511; thereby increasing, decreasing or ceasing thermoregulation.

Optionally refrigerated unit 500 includes a visual display 518 that displays the loss of calories based upon, for example: the temperature of unit 500, user weight, body temperature, and the period of use by the user noted above.

Visual display 518 optionally includes red LED's 520 and blue LED's 521 that prompt the user in entering information by pressing red LED 520 and/or blue LED 521. Alternatively, LED's 520 and LED's 521 comprise any color; the many possible colors of LED's 520 and LED's 521 being well known to those familiar with the art.

Alternatively, the user presses a button associated with red LED 520 and/or blue LED 521 located on a chair armrest 517 in response to queries on visual display 518.

For example, the user is queried on display 518 whether to lower the temperature of refrigerated unit 500. By pressing a button displaying the word “yes” and associated with red LED 520, the user indicates “yes”. Pressing a button displaying the word “no” and associated with blue LED 521 indicates “no”.

Computerized Peltier Unit

FIG. 4C shows a chair comprising a computerized Peltier pump system 526 that cools and circulates fluid through circulation tubes 542 on a seat 524.

Computerized Peltier system 526 optionally includes a touch screen 530, which prompts the user to indicate user preferences, for example, the temperature of fluid through circulation tubes 542. In addition, touch screen 530 provides ongoing information to the user regarding, inter alia, caloric output, body temperature, heart rate, and respiration rate.

Optionally, touch screen 530 and a state-of-the-art “surround sound” speaker system 516 that wirelessly interfaces with computer 482. Optionally, the user programs computer 482 with sound and visual software that optionally aid in caloric loss of the user. For example, via software in computer 482, touch screen 530 optionally shows the user exercises to perform with upper and/or lower extremities while “surround sound” speaker system 516 plays music that provides an appropriate beat and instrumentation to stimulate the user to perform the exercises.

Optionally, the color patterns are provided on a rhythmic basis that promotes the user to breath faster or slower in order to enhance caloric loss.

Additionally or alternatively, touch screen 530 shows the user color patterns associated with fluid temperature within circulation tubes 542. For example, darker colors signify colder temperatures and lighter colors signify warmer temperatures.

In alternative embodiments, touch screen 530 and “surround sound” speaker system 516 wirelessly interface with a satellite dish 548 so that the user, can work while sitting on unit 500.

In further embodiments, computerized Peltier system 526 optionally includes an energy conservation sensor 522, which senses the weight of a user and automatically turns off computerized Peltier system 526 after a period of time following after the user gets leaves thermoregulating unit 540.

In further embodiments, computer 482 is programmed with a seasonally adjusted energy saver mode that programs computerized Peltier system 526 to conserve energy at peak energy consumption hours.

In non-limiting embodiments, computerized Peltier system 526 is located in a chair base 562, which includes air channels 540 that allow air to circulate through chair base 562 and exchange heat with Peltier pump 526.

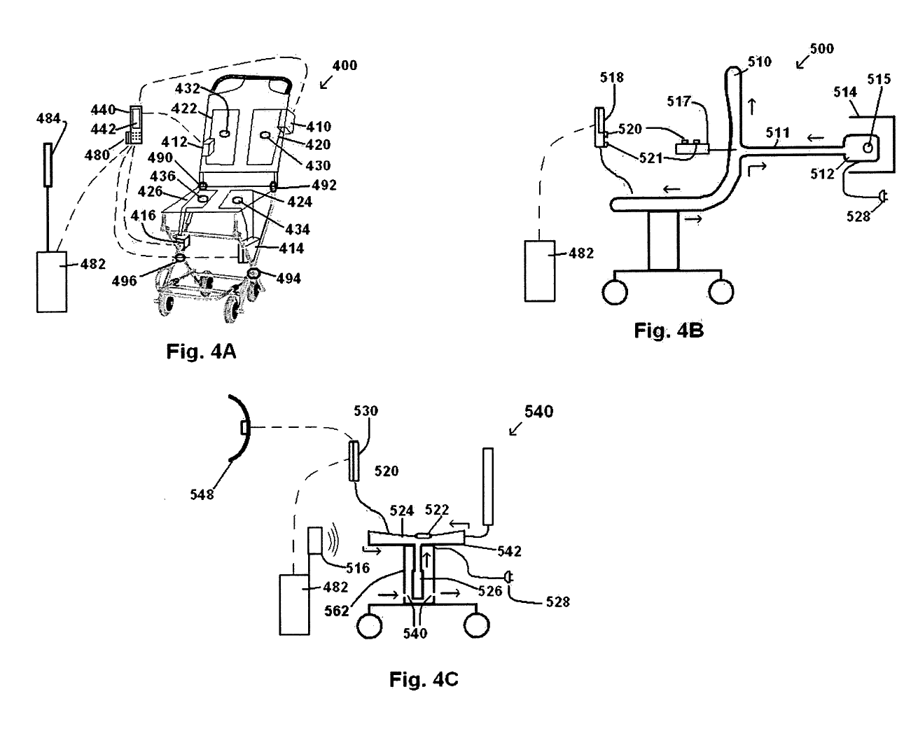

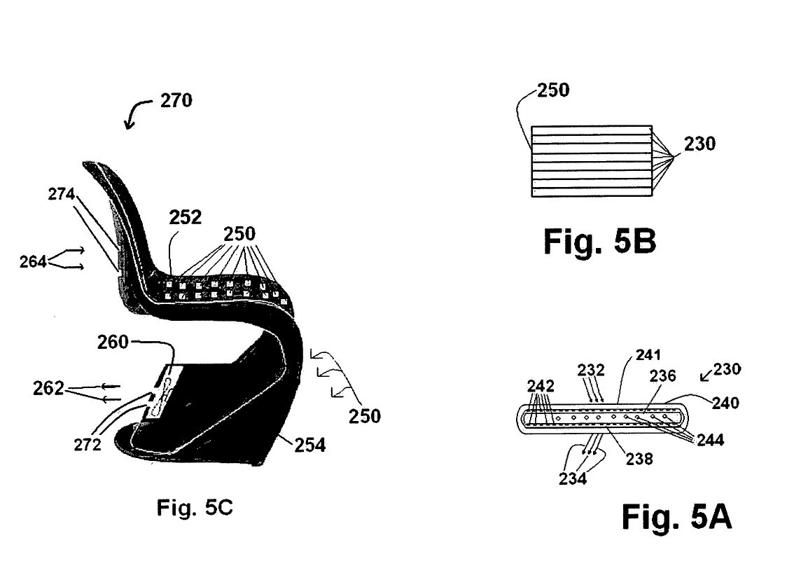

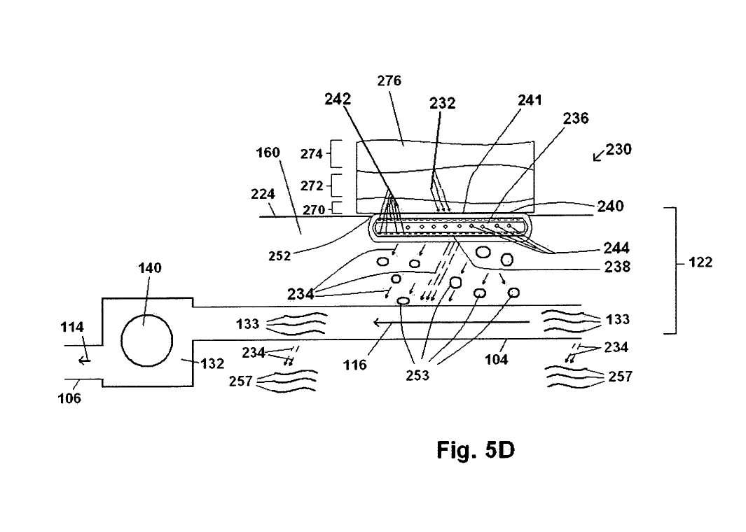

Alternatively, as noted in unit 270 (FIG. 5C) below, fan 260 facilitates heat exchange.

Heat Pipes and Mega Flats

FIG. 5A shows a heat pipe 230 consisting of a sealed metal container 240, comprising, for example aluminum or copper. Lining container 240 is a wicking material 238 and within wicking material 238 is a liquid 236 under its own pressure that enters wicking material pores 242, thereby wetting wicking material 238.

A heat input 232, along any point of the upper surface of heat pipe 230 causes liquid 236 to boil and enter a vapor state, becoming a gas 244. When that happens, liquid 236 picks up the latent heat associated with gas 244 and gas 244, which has a higher pressure, moves inside sealed container 240 to a colder location and away from body where it condenses.

In this manner, gas 244 gives up the latent heat of vaporization and moves heat from heat input 232 to a heat output 234 along the lower (as pictured) surface of heat container 240 where the heat comes into contact with an environment that is conducive to removing heat from container 240.

Heat pipes 230 are known to have an effective thermal conductivity that may be tens, hundreds or even thousands of times that of copper.

Heat pipes 230 are optionally manufactured to maintain a constant temperature or temperature range.

In embodiments, heat pipes 230 are connected together longitudinally to form what is referred to as a mega flat 250, as seen in FIG. 5B; an example of which is manufactured by Noren Products Inc of Menlo Park, Calif., USA.

Mega flats 250 may be manufactured in almost any size and shape including a flat shape. If range of motion is required, mega flats 250 may be optionally made of flexible material. Flat and flexible mega flats 250 may be desirable, for example, for embedding and/or attaching to an upholstery surface that is in contact with the user to provide effective thermoregulation during contact.

FIG. 5C shows a chair comprising an ergonomic unit 270 having multiple mega flats 250 embedded in, and/or attached to, the surface of a seating area 252 and a leg rest area 254. Leg rest area 254 is contoured so that the user has the option to press against or remove one or both legs from multiple mega flats 250 embedded in the upholster.

In embodiments, ergonomic unit 270 includes a fan 260 that causes cool air 264 to enter ergonomic unit 270, exchange heat with mega flats 250. The heated air exits ergonomic unit 270 through rear seat back openings 274, with the circulation of air against mega flats 250 acting as a heat sink to facilitate a comfortable temperature in ergonomic unit 270.

Exemplary Passive Thermoregulation Detail

FIG. 5D shows heat flow 234 from a portion of skin 276, comprising a hypodermis 274, an endodermis 272 and an epidermis 270 in contact with thermoregulating seat 122.

Cooling fluid 133 releases heat output 234 to air 257 in contact with inlet tube 104. In this manner, cooling fluid 133 acts to passively remove heat from thermoregulating chamber 160 thereby maintaining a continual rate of heat loss and/or comfort level of skin portion 276.

Alternatively, external pump 132 pumps cooling fluid 133 through cooling unit 102 (FIG. 1) where heat output 234 is actively removed from fluid 133, thereby actively reducing the temperature of cooling fluid 133.

The inventors believe that some embodiments of the present invention presented herein have the potential to reduce body metabolism in a manner that facilitates weight loss; weight loss that potentially occurs substantially without the pain and/or the discomfort associated with shivering in the above-noted article: “Thermoregulatory Thermogenesis of Humans During Cold Stress”.

The inventions have described herein include a number of features that are now summarized:

Quiet

Embodiments of the thermoregulating units of the present invention are optionally designed to perform quietly, for example with the addition of acoustic noise dampener 134 seen in FIG. 1.

Comfort

Further Embodiments of the thermoregulating units of the present invention can optionally be designed to comfortably embrace the user as seen in unit 270 depicted in FIG. 5C.

Simple Technology

Additionally, embodiments of the thermoregulating units of the present invention optimally use simple devices to effectively promote thermal exchange, for example exhaust fan 260, as depicted in FIG. 5C.

Additionally, embodiments of the thermoregulating units of the present invention are easily used with modern conveniences, as seen in cooling bars 212 in FIG. 2 that can be cooled in a refrigerator or freezer.

Readily Adaptable

Embodiments of the thermoregulating units of the present invention are readily adaptable to many environments, for example being easily configured for use in vehicle 318 shown in FIG. 3.

Computer Features

In addition, embodiments of the thermoregulating units of the present invention benefit from the computer age. For example, unit 500 depicted in FIG. 4B includes a personal display 518 of calorie loss, while unit 540 of FIG. 4C includes “surround sound speakers” 516 for entertainment and satellite dish 548 for business needs.

Safe

Embodiments of the thermoregulating units of the present invention include safety features, for example sensors 430 and 432 on unit 400 that prevent thermal changes from reaching intolerable and/or dangerous levels.

Personalized

Moreover, Embodiments of the thermoregulating units of the present invention allow personalization of thermoregulation agendas, for example using inputs 520 and 521 on unit 500 (FIG. 4B) for the user to signal change in a given thermotherapy regimen.

Environmentally Friendly

In further Embodiments, the thermoregulating units of the present invention, include environmentally friendly features, for example limiting the cycling of the thermoregulating units during peak energy levels usage in a city.

It is expected that during the life of this patent many relevant cooling technologies will be developed and the scope of the terms cooling technologies, cooling units, and cooling bars are intended to include all such new technologies a priori.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art.

Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A thermoregulating unit for increasing metabolism of at least a portion of a human body, the thermoregulating unit comprising:
a support surface configured for supporting at least a portion of a human body; and
at least one thermoregulating chamber juxtaposed on said support surface, said at least one thermoregulating chamber configured to draw heat away from said at least a portion of the human body supported on said support surface.

2. The thermoregulating unit according to claim 1, wherein said at least one thermoregulating chamber passively draws heat away from said at least a portion of the human body supported on said support surface.

3. The thermoregulating unit according to claim 1, wherein said at least one thermoregulating chamber is removably attached to said support surface.

4. The thermoregulating unit according to claim 3, wherein said at least one thermoregulating chamber is configured to be cooled in a cooling unit.

5. The thermoregulating unit according to claim 1, wherein said support surface comprises at least one of:
one seat;
one backrest;
one headrest;
one armrest;
one hand rest;
one leg rest; and
one footrest.

6. The thermoregulating unit according to claim 1, wherein said at least one thermoregulating chamber includes a foam material.

7. The thermoregulating unit according to claim 6, wherein said foam material comprises an open cell thermoregulating foam material.

8. The thermoregulating unit according to claim 7, wherein said foam material includes thermo-conductors from the group of thermo-conductors consisting of: metal pads, liquid capsules, diamond chips, heat pipes, and mega flats.

9. The thermoregulating unit according to claim 8, wherein said at least one thermoregulating chamber includes a fan configured to foster heat exchange between said thermo-conductors and the environment.

10. The thermoregulating unit according to claim 9 wherein said at least one thermoregulating chamber includes at least one removable cooling insert.

11. The thermoregulating unit according to claim 10, wherein said at least one cooling insert contains at least one of a phase-change liquid and a chemically reactive material.

12. The thermoregulating unit according to claim 1, wherein at least a portion of said thermoregulating supporting surface is contoured to at least partially curve around at least a portion of a human body.

13. The thermoregulating unit according to claim 1, wherein said at least one thermoregulating chamber includes apertures configured to allow passage of gas from the surrounding environment into said one chamber.

14. The thermoregulating unit according to claim 13, wherein said apertures are configured to allow passage of a gas from said at least one thermoregulating chamber to the surrounding environment.

15. The thermoregulating unit according to claim 13, configured for use in a vehicle.

16. The thermoregulating unit according to claim 15, wherein passage of said gas is in response to movement by said vehicle.

17. The thermoregulating unit according to claim 1, including:
i) a tubular portion connected to said support surface, said tubular containing a circulating cooling fluid; and
ii) a pump connected to said tubular portion and configured to circulate said cooling fluid through said tubular portion.

18. The thermoregulating unit according to claim 17, including a controller adapted to actively control a rate at which said pump circulates said cooling fluid.

19. The thermoregulating unit according to claim 18, including at least one sensor connected to said controller, said sensor measuring the temperature in said at least one body portion.

20. The thermoregulating unit according to claim 19, wherein said controller is configured to provide greater cooling when said at least one body portion is above the ambient temperature.

21. The thermoregulating unit according to claim 20, including one vibrator operatively associated with said unit.

22. The thermoregulating unit according to claim 21, wherein said one vibrator is configured to cause frictional interaction between said portion of the human body and said support surface, thereby causing vasodilation that increases a rate of heat loss.

23. The thermoregulating unit according to claim 17, wherein said cooling unit is from the group of cooling units consisting of a Peltier unit, and an evaporative cooling unit, a compressor-activated cooling unit, a fluid expansion cooling unit, a compressed fluid cooling unit a Karno machine an absorption cooler, and a Stirling engine.

24. The thermoregulating unit according to claim 17, wherein said cooling unit is powered by a power supply from the group consisting of a self-contained battery, a car battery, and an alternating current.

25. The thermoregulating unit according to claim 17, including acoustic insulation at least partially surrounding said cooling unit.

26. The thermoregulating unit according to claim 17, including a noise canceling wave generator operatively associated with said cooling unit.

27. The thermoregulating unit according to claim 17, including at least one sensor configured to measure one parameter of the at least one body portion from the group of parameters comprising user weight, body temperature, caloric output, heart rate, and respiration rate.

28. The thermoregulating unit according to claim 27, including a controller connected to said sensor, said controller adapted to actively control a rate at which cooling fluid circulates based upon said one parameter.

29. The thermoregulating unit according to claim 28, wherein said sensor is configured to sense when the user has left the thermoregulating unit.

30. The thermoregulating unit according to claim 28, wherein said controller is configured to sense when a user has left said unit and reduce power consumption of said unit.

31. The thermoregulating unit according to claim 28, wherein said controller is programmed with a seasonally adjusted energy saver mode that automatically conserves energy consumption at peak energy consumption hours by regulating at least one of: said cooling unit; and a fluid pump.

32. A method for increasing metabolism of a subject's body in order to lose weight, comprising:
a) supporting a part of the body on a thermoregulating supporting surface; and
b) thermoregulating said part of said body to increase the metabolism of said part.

33. The method according to claim 32, including placing said thermoregulating supporting surface in a vehicle.

34. The method according to claim 32, including cooling said thermoregulating supporting surface to increase the metabolism of said part.

35. The method according to claim 32, including heating said thermoregulating supporting surface to increase the metabolism of said part.

36. The method according to claim 32, including vibrating said thermoregulating supporting surface to increase the metabolism of said part.
